(19) United States
(12) Patent Application Publication
(10) Pub. No.: US 2005/0123595 A1
(43) Pub. Date: Jun. 9, 2005

(54) Composition for removing tattoos

(76) Inventors: J. Kampinga, Berlin (DE); S. Stienstra, Berlin (DE)

Correspondence Address: SMITH PATENT OFFICE, 1901 PENNSYLVANIA AVENUE N W, SUITE 200, WASHINGTON, DC, 20006, US

(21) Appl. No.: 10/938,872

(22) Filed: Sep. 13, 2004

(30) Foreign Application Priority Data
Sep. 11, 2003 (EP) 03077869

(51) Int. Cl. A61K 9/127; A61K 38/00; A61N 1/30

(52) U.S. Cl. 424/450; 514/2; 604/20

(57) ABSTRACT

A composition and method for removing tattoos is disclosed. The method includes administering a composition including a photosensitiser to the skin area comprising the tattoo, allowing the histiocytes in the dermis of the tattoo area to take up the phtosensitiser, and illuminating the treated skin area to induce production of singlet oxygen for killing the histiocytes that have taken up the photosensitiser.

The present invention relates to a composition for removing tattoo's by using photosensitisers and to a method for doing same.

The tattoo is very popular with young people. However, it is also known that many of them regret the tattoo in later years and want it removed. The current practice is to treat the tattoo with a laser. The disadvantage is that the treatment often leads to scar tissue and that the melanocytes in the treated area may be killed. This may lead to a pale area in the skin where the tattoo has been.

The invention has for its object to provide an alternative treatment that avoids the above described disadvantages.

The invention is based on the idea that ink that is used for tattoo's consists of little particles. Most of these particles are being taken up rapidly by tissue macrophages in the dermis. These so-called histiocytes can not destroy the ink particles and because histiocytes have a very low turnover in the dermis, a tattoo can stay for a very long time visible.

The present inventors considered to put a photosensitiser on top of the skin overlaying the tattoo and then to use a skin penetration enhancer to get the photosensitiser through the epithelial layer into the dermis. Upon illumination the photosensitiser can then bleach the ink and/or damage the cells containing the ink particles. In another embodiment the photosensitisers are injected directly into the dermis in and around the tattoo. By using the appropriate photosensitiser in combination with the appropriate light it is thus possible to remove the tattoo.

In a still further embodiment, photosensitisers (or substances that create photosensitisers inside histiocytes) can be bound to little particles. Injecting them through the epidermis by using appropriate means (such as tattoo gun, fluid jet apparatus or needles with a syringe) at the same location as the tattoo, will cause the histiocytes to take up these particles. After such treatment, appropriate light will activate the photosensitiser and damage specifically the histiocytes in the tattoo area. This will release the ink particles from the histiocytes, which will subsequently be transported from the tissue fluid to the lymph nodes. The killed histiocytes will get replaced by fresh histiocytes that do not contain ink particles. This treatment protocol with particles is more efficient, specific and less damaging for the dermis tissue in the tattoo.

The invention thus relates to a composition for removing tattoo's, comprising a photosensitiser.

The composition can take the form of a liquid, gel or cream. This may be incorporated in a kind of bandage such as plasters. Preferably, the treatment in which such composition is used also comprises a skin penetration enhancing method, such as by chemical or physical means.

In another embodiment the composition comprises particles of the photosensitiser that can be taken up by histiocytes. The photosensitiser may be absorbed on, coated on or incorporated in solid, porous or colloidal particles. Many types of inorganic or organic particles could be used for this purpose, such as metals, polymers, ceramics, glass and carbohydrate (derivative)s. Alternatively, the photosensitiser constitutes the particles or the photosensitiser is complexed with proteins (such as human albumin), lipids or other biological materials that are formed into particles.

Such particles may also consist of vesicles, including liposomes, containing the photosensitiser inside and/or coated on and/or as part of the vesicle wall-material. Vesicles with a preferential affinity to histiocytes compared to the other cells and structures of the dermis are preferred.

The invention further relates to a method for removing tattoo's from skin, comprising administering the composition to the skin area comprising the tattoo, allowing the histiocytes in the dermis of the tattoo area to take up the photosensitiser, and illuminating the treated skin area to induce production of singlet oxygen for killing the histiocytes that have taken up the photosensitiser. The method may further comprise a skin penetration enhancing method, such as by chemical or physical means.

1. A composition for removing a tattoo, comprising a photosensitiser.

2. A composition as claimed in claim 1, wherein the composition takes the form of a liquid, gel or cream.

3. A composition as claimed in claim 1, wherein the composition comprises particles comprising the photosensitiser that can be taken up by histiocytes.

4. A composition as claimed in claim 3, wherein the photosensitiser is absorbed on, coated on or incorporated in the particles.

5. A composition as claimed in claim 4, wherein the particles are solid, porous or colloidal.

6. A composition as claimed in claim 3, wherein the photosensitiser constitutes the particles.

7. A composition as claimed in claim 3, wherein the photosensitiser is complexed with proteins, lipids or other biological materials that are formed into particles.

8. A composition as claimed in claim 3, wherein the photosensitiser is contained inside and/or coated on and/or being part of the wall material of vesicles, including liposomes.

9. A composition as claimed in claim 8, wherein the vesicles have a preferential affinity to histiocytes compared to the other cells and structures of the dermis.

10. A method for removing a tattoo from skin, comprising the steps of:
administering a composition including a photosensitiser to the skin area comprising the tattoo, allowing the histiocytes in the dermis of the tattoo area to take up the phtosensitiser, and illuminating the treated skin area to induce production of singlet oxygen for killing the histiocytes that have taken up the photosensitiser.

11. A method as claimed in claim 10, further comprising a skin penetration enhancing method, such as by chemical or physical means.

12. A method as claimed in claim 10, wherein the composition in said step of administering takes the form of a liquid, gel or cream.

13. A method as claimed in claim 10, wherein the composition in said step of administering comprises particles comprising the photosensitiser that can be taken up by histiocytes.

14. A method as claimed in claim 13, wherein the photosensitiser in said step of administering is absorbed on, coated on or incorporated in the particles.

15. A method as claimed in claim 14, wherein the particles in said step of administering are solid, porous or colloidal.

16. A method as claimed in claim 13, wherein the photosensitiser in said step of administering constitutes the particles.

17. A method as claimed in claim 13, wherein the photosensitiser in said step of administering is complexed with proteins, lipids or other biological materials that are formed into particles.

18. A method as claimed in claim 13, wherein the photosensitiser in said step of administering is contained inside and/or coated on and/or being part of the wall material of vesicles, including liposomes.

19. A method as claimed in claim 18, wherein the vesicles in said step of administering have a preferential affinity to histiocytes compared to the other cells and structures of the dermis.
